(19) United States
(12) Patent Application Publication
(10) Pub. No.: US 2012/0114720 A1
(43) Pub. Date: May 10, 2012

(54) SOLID, FRAGRANCE-TRANSMITTING COMPOSITION HAVING GOOD COLD-WATER SOLUBILITY

(75) Inventors: Theodor Völkel, Erkrath (DE); Hans Bouten, Geldern (DE); Dietmar Schröter, Dusseldorf (DE); Tatiana Schymitzek, Krefeld (DE)

(73) Assignee: Henkel AG & Co. KGaA, Dusseldorf (DE)

(21) Appl. No.: 13/349,752

(22) Filed: Jan. 13, 2012

Related U.S. Application Data
(63) Continuation of application No. PCT/EP2010/059464, filed on Jul. 2, 2010.

(30) Foreign Application Priority Data
Jul. 16, 2009 (DE) 10 2009 027 756.0

(51) Int. Cl. A61K 8/02; A61Q 19/00; B05D 7/00; C11D 3/60

(52) U.S. Cl. 424/401; 512/4; 510/516; 510/528; 510/530; 510/513; 427/212

(57) ABSTRACT

The invention relates to a solid fragrance-transmitting composition, comprising a particulate, water-soluble carrier, a perfume, and at least partial shell made of a saccharide. The solid fragrance-transmitting composition is very soluble in cold water and is thus suitable for use in the rinse cycle of a washing machine.

### CROSS-REFERENCES TO RELATED APPLICATIONS

This application is a continuation of PCT/EP2010/059464, filed on Jul. 2, 2010, which claims priority under 35 U.S.C. §119 to DE 10 2009 027 756.0 filed on Jul. 16, 2009, both of which are hereby incorporated by reference.

### FIELD OF THE INVENTION

The present invention generally relates to a solid fragrance-transmitting composition as well as to its use and manufacture.

### BACKGROUND OF THE INVENTION

Repeated washing of textiles often causes them to become hard and to lose their softness. In order to restore their softness/flexibility, to lend them a pleasant fragrance and/or to improve their antistatic properties, the textiles, after the actual washing and cleaning process, are treated with a fabric softener in a subsequent rinse process.

The majority of the commercial fabric softeners are aqueous formulations that comprise a cationic fabric-softening compound having one or two long chain alkyl groups in a molecule as the major active ingredient. Common cationic fabric-softening compounds include for example methyl-N-(2-hydroxyethyl)-N,N-di(tallowacyloxyethyl)ammonium compounds or N,N-dimethyl-/N,N-di-(tallowacyloxyethyl)ammonium compounds.

A disadvantage of these conventional fabric softeners is that they frequently leave an unsightly residue in the dispensing draw of the washing machine. In addition, the conventional fabric softeners do not prevent the deposition of lime scale residues on the washing during the rinse cycle.

In many cases, for example with towels, the use of a fabric softener is undesirable or even unsuitable. Many consumers do not use any fabric softeners for towels as the contained esterquats reduce the absorbency and water uptake of the towels.

Nevertheless, consumers would also like the washing and/or rinsing to transmit a pleasant fragrance to these textiles.

Solid, fragrance-transmitting and fabric-care compositions having a crystal-like visual appearance which include a water-soluble carrier, a water-soluble polymer, a fabric-care compound and a perfume are known from WO 2007/115872 A1.

These fragrance-transmitting and fabric-care compositions are added in the main wash cycle together with washing or cleaning agents. Esthetically particularly attractive solid fragrance-transmitting and fabric-care compositions are obtained with polyalkylene glycols, particularly polyethylene glycol, as the water-soluble polymer.

However, many consumers want a solid fragrance-transmitting composition that can be added only in the last rinse cycle of a washing machine and is metered through the dispensing draw for washing agents or conditioners. The solid fragrance-transmitting compositions known from WO 2007/115872 A1 are indeed water-soluble, but they do not fulfill the requirement of dissolving rapidly in cold water. However, this is necessary for an unproblematic metering through the dispensing draw for washing agents or conditioners. This is all the more true in view of the fact that modern washing machines use less and less water for the washing and rinsing processes.

Accordingly, it is desirable to provide a fragrance-transmitting composition having a crystalline-like visual appearance which can be added in the last rinse cycle of a washing machine.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

One aspect of the present invention relates to a solid fragrance-transmitting composition comprising a particulate water-soluble carrier, and perfume, wherein the composition has at least partial coating of a saccharide, wherein the saccharide is selected from the group of monosaccharides, oligosaccharides and mixtures thereof.

The invention also relates to the method of using of an inventive solid fragrance-transmitting composition for conditioning textile fabrics.

A further aspect of the present invention includes process for manufacturing a solid fragrance-transmitting composition comprising a particulate water-soluble carrier, and perfume, wherein the composition has at least partial coating of a saccharide, wherein the saccharide is selected from the group of monosaccharides, oligosaccharides and mixtures thereof, in which the water-soluble carrier is treated with the perfume, the saccharide is melted and is deposited in such a way onto the particulate carrier such that said carrier is at least partially coated.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A rapid (cold) water-soluble coating is obtained by using monosaccharides and/or oligosaccharides as the coating material, which does not negatively impair the visual appearance of the solid fragrance-transmitting composition.

Moreover, this solid composition is easier and better to handle than the liquid compositions as no drops remain on the rim of the bottle, which during subsequent storage of the bottle lead to marks underneath or to unsightly deposits in the area of the closure. The same is true for the case when some of the composition is accidentally spilled on being dosed. The spilled composition can also be removed more easily and cleanly.

The water-soluble carrier is preferably selected from the group consisting of inorganic alkali metal salts, organic alkali metal salts, inorganic alkaline earth metal salts, organic alkaline earth metal salts, organic acids, carbohydrates, silicates, urea and mixtures thereof.

These materials are not only inexpensive but dissolve very well in water. Moreover, these materials are odorless.

The amount of water-soluble carrier is advantageously 50 to 99 wt %, preferably 75 to 95 wt %, each based on the total solid fragrance-transmitting composition.

It is further preferred that the solid fragrance-transmitting composition additionally possesses an ingredient selected from the group of the fabric-care compounds, colorants, fillers, pearlizers, skin-care compounds and mixtures thereof.

The fabric-care compound can be selected from fabric-softening compounds, bleaching agents, bleach activators, enzymes, silicone oils, anti-redeposition agents, optical brighteners, graying inhibitors, shrink inhibitors, anti-creasing agents, color transfer inhibitors, antimicrobials, germicides, fungicides, antioxidants, antistats, ironing auxiliaries, water proofing and impregnation agents, swelling and anti-slip agents, UV absorbers as well as mixtures thereof.

By adding fabric-care compounds for example, the solid fragrance-transmitting composition can be easily provided with additional functionalities.

It is particularly preferred that the fabric-care compound is a fabric-softening compound. In this regard it is quite particularly preferred that the fabric-softening compound is selected from quaternary ammonium compounds, polysiloxanes, fabric-softening clays, cationic polymers and mixtures thereof.

The use of polysiloxanes and/or cationic polymers as the fabric-care compound in the fragrance-transmitting composition is advantageous, as they not only show a softening effect but also reinforce the impression of the perfume on the washing The use of softening clays as the fabric-care compound in the fragrance-transmitting composition is advantageous, as they additionally show a water-softening effect and thus lime scale deposits on the laundry are prevented. In order to achieve an optimal performance, it can be preferred that a fragrance-transmitting composition comprises a combination of at least two fabric-care compounds. The choice of fabric-softening compound also depends on the intended use of the fragrance-transmitting composition. Thus, it may be appropriate for the solid fragrance-transmitting composition to be free of cationic, fabric-softening compounds.

It is particularly preferred that the saccharide is saccharose. Saccharose exhibits a high solubility of 2.4 g per g water at 20 ° C.

In a preferred embodiment, the fragrance-transmitting composition comprises 0.1 to 20 wt %, preferably 1 to 10 wt % and particularly preferably 2 to 7 wt % perfume.

For conventional liquid, fabric-softener compositions containing quaternary ammonium compounds, a problem also arises with the stability of the composition at higher perfume concentrations (>0.4 wt % perfume in regular fabric-softener compositions and >1 wt % in concentrated fabric-softener compositions). Higher amounts (>1 wt %) of perfume can be incorporated without difficulty into the fragrance-transmitting compositions according to the invention.

In a particularly preferred embodiment of a fragrance-transmitting and fabric-care composition, the fabric-care compound is in powder form and is present at least partially in and/or at least partially on the coating.

In this preferred embodiment it can be advantageous when a polysaccharide is present at least partially in and/or at least partially on the coating, in addition to the fabric-care compound.

In all embodiments of the fragrance-transmitting composition, it is preferred that the particle sizes of the water-soluble carrier are in the range 0.6 to 30 mm, particularly 0.8 to 7 mm and particularly preferably 1 to 3 mm.

Fragrance-transmitting compositions with particle sizes in the range 0.8 to 7 mm and particularly preferably in the range 1 to 3 mm can be dosed particularly well and in a controlled manner.

In order to improve the application and/or esthetic properties of the solid fragrance-transmitting composition, it can comprise additional ingredients, preferably selected from the group consisting of colorants, perfume, fillers, pearlizers, skin-care compounds and mixtures thereof.

The invention is described below in more detail inter alia by means of examples.

The solid fragrance-transmitting composition comprises a particulate, water-soluble carrier, a water-soluble saccharide and a perfume as the essential ingredients.

One essential ingredient of the fragrance-transmitting composition is the water-soluble carrier. This preferably includes inorganic alkali metal salts such as for example sodium chloride, potassium chloride, sodium sulfate, sodium carbonate, potassium sulfate, potassium carbonate, sodium hydrogen carbonate, potassium hydrogen carbonate or mixtures thereof, organic alkali metal salts such as for example sodium acetate, potassium acetate, sodium citrate, sodium tartrate or potassium sodium tartrate, inorganic alkaline earth metal salts such as for example calcium chloride or magnesium chloride, organic alkaline earth metal salts such as for example calcium lactate, carbohydrates, organic acids such as for example citric acid or tartaric acid, silicates such as for example water glass, sodium silicate or potassium silicate, urea as well as mixtures thereof. The water-soluble carrier can contain in particular a carbohydrate that is selected for example from the group consisting of dextrose, fructose, galactose, isoglucose, glucose, saccharose, raffinose, isomalt and mixtures thereof. The added carbohydrate can be for example candy sugar or coarse sugar.

As carriers, carbohydrates have the advantage that they do not promote the corrosion of metallic parts in automatic washing machines. Citric acid has the advantage as a carrier in that the fragrance-transmitting composition simultaneously possesses a builder for reducing the water hardness.

The water-soluble carrier can also comprise mixtures of the cited materials.

Preferably, the water-soluble carrier is particulate and has particle sizes in the range 0.6 to 30 mm, particularly 0.8 to 7 mm and particularly preferably 1 to 3 mm.

Another ingredient of the fragrance-transmitting composition is the water-soluble saccharide that at least partially coats the water-soluble carrier. The saccharide is selected from the group of the monosaccharides, the oligosaccharides and mixtures thereof.

Monosaccharides are linear polyhydroxyaldehydes (aldoses) or polyhydroxyketones (ketoses) and preferably include pentoses (C₅H₁₀O₅) and hexoses (C₆H₁₂O₆). Exemplary suitable pentoses are xylose, lyxose, ribose and arabinose. Exemplary suitable hexoses include glucose, galactose or mannose.

Oligosaccharides are formed by coupling 2 to 10 monosaccharide molecules to larger molecules under water-elimination, and include disaccharides, trisaccharides, etc. Exemplary suitable oligosaccharides are saccharose, lactose, maltose. The saccharide saccharose is particularly preferably employed.

The water-soluble saccharide can also comprise a mixture of the cited mono and/or oligosaccharides.

Another essential ingredient of the fragrance-transmitting composition is the perfume. Suitable perfume oils or fragrances include individual odoriferous compounds, for example synthetic products of the ester, ether, aldehyde, ketone, alcohol and hydrocarbon type. However, mixtures of various odoriferous substances, which together produce an attractive fragrant note, are preferably used. Such perfume oils can also comprise natural mixtures of odoriferous compounds, as are available from vegetal sources.

Preferably, the quantity of perfume in the fragrance-transmitting composition ranges between 0.1 and 20 wt %, particularly preferably between 1 and 10 wt % and quite particularly preferably between 2 and 7 wt %.

The fragrance-transmitting composition can optionally comprise additional ingredients that are selected from the group of the fabric-care compounds, colorants, fillers, pearlizers, skin-care compounds and mixtures thereof.

In this context, a fabric-care compound is understood to mean each compound that provides a beneficial effect to textile fabrics that are treated with it, such as for example a fabric-softening effect, crease resistance, or that reduces the damaging or negative effects that can arise on cleaning and/or conditioning and/or wearing, such as for example fading, graying, etc.

The fabric-care compound can contain for example a fabric-softening compound, bleaching agents, bleach activators, enzymes, silicone oils, anti-redeposition agents, optical brighteners, graying inhibitors, shrink inhibitors, anti-creasing agents, color transfer inhibitors, antimicrobials, germicides, fungicides, antioxidants, antistats, ironing auxiliaries, water proofing and impregnation agents, swelling and anti-slip agents, UV absorbers and mixtures thereof.

The fabric-care compound is preferably a fabric-softening compound and is for example a quaternary ammonium compound, a polysiloxane, a fabric-softening clay, a cationic polymer or a mixture of at least two of these fabric-care compounds. Accordingly, the fragrance-transmitting composition is preferably a fabric-softening composition.

Preferred employable quaternary ammonium compounds are esterquats. Suitable examples of esterquats are shown for example in the Formulas (I) and (II):

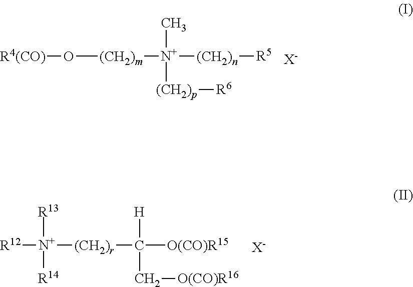

In Formula (I), R⁴ stands for an aliphatic alk(en)yl group containing 12 to 22 carbon atoms with 0, 1, 2 or 3 double bonds and/or optionally with substituents; R⁵ stands for H, OH or O(CO)R⁷, R⁶ independently of R⁵ stands for H, OH or O(CO)R⁸, wherein R⁷ and R⁸, independently of each other, each stand for an aliphatic alk(en)yl group having 12 to 22 carbon atoms with 0, 1, 2 or 3 double bonds, m, n, and p independently of each other can each have the value 1, 2 or 3. X³¹ can be either a halide ion, methosulfate ion, methophosphate ion or phosphate ion as well as mixtures of these anions. Compounds are preferred in which R⁵ represents the group O(CO)R⁷. Compounds are particularly preferred in which R⁵ represents the group O(CO)R⁷ and R⁴ and R⁷ are alk(en)yl groups with 16 to 18 carbon atoms.

Compounds in which R⁶ stands moreover for OH are particularly preferred. In Formula (II), R¹², R¹³ and R¹⁴ independently of one another stand for a C₁₋₄ alkyl, alkenyl or hydroxyalkyl group, R¹⁵ and R¹⁶, each independently selected, represents a C₈₋₂₈ alkyl group, X⁻ is an anion and r is a number between 0 and 5. Examples of usable esterquats include methyl-N-(2-hydroxyethyl)-N,N-di(tallowacyloxyethyl)ammonium methosulfate, bis-(palmitoyloxyethyl)-hydroxyethyl-methylammonium methosulfate, 1,2-bis-[tallowacyloxy]3-trimethylammonium propane chloride, methyl-N,N-bis(stearoyloxyethyl)-N-(2-hydroxyethyl)ammonium methosulfate or N,N-dimethyl-N,N-di(tallowacyloxyethyl)ammonium methosulfate.

A preferred useable polysiloxane possesses at least the following structural units

a)

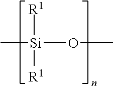

with

R¹=independently of one another C₁-C₃₀ alkyl, preferably C₁-C₄ alkyl, in particular methyl or ethyl,

n=1 to 5000, preferably 10 to 2500, in particular 100 to 1500.

It can be preferred that the polysiloxane also possesses the following additional structural units:

b)

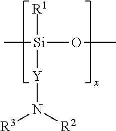

with

R¹=C₁-C₃₀ alkyl, preferably C₁-C₄ alkyl, especially methyl or ethyl,

Y=optionally substituted, linear or branched C₁-C₂₀ alkylene, preferably —(CH₂)ₘ— with m=1 to 16, preferably 1 to 8, in particular 2 to 4, especially 3,

R², R³=independently of one another H or optionally substituted, linear or branched C₁-C₃₀ alkyl, preferably C₁-C₃₀ alkyl substituted with amino groups, particularly preferably —(CH₂)_{b}—NH₂ with b=1 to 10, most preferably b=2,

x=1 to 5000, preferably 10 to 2500, in particular 100 to 1500.

If the polysiloxane only possesses the structural unit a) with R¹=methyl, then it is a polydimethylsiloxane. Polydimethylpolysiloxanes are known as efficient fabric-care compounds.

Suitable polydimethysiloxanes include DC-200 (ex Dow Corning), Baysilone® M 50, Baysilone® M 100, Baysilone® M 350, Baysilone® M 500, Baysilone® M 1000, Baysilone® M 1500, Baysilone® M 2000 or Baysilone® M 5000 (all ex GE Bayer Silicones).

It can also be preferred that the polysiloxane comprises the structural units a) and b). A particularly preferred polysiloxane possesses the following structure:

(CH₃)₃Si—[O—Si(CH₃)₂]ₙ—[O—Si(CH₃){CH₂)₃—NH—(CH₂)₂—NH₂}]ₓ—OSi(CH₃)₃

wherein the sum of n+x is a number between 2 and 10 000.

Suitable polysiloxanes with the structural units a) and b) are commercially available for example under the brand names DC2-8663, DC2-8035, DC2-8203, DC05-7022 or DC2-8566 (all ex Dow Corning). The commercially available products Dow Corning® 7224, Dow Corning® 929 Cationic Emulsion or Formasil 410 (GE Silicones) are likewise suitable according to the invention.

An exemplary suitable fabric softener clay is a smectite clay. Preferred Smectite clays are Beidellite clays, Hectorite clays, Laponite clays, Montmorillonite clays, Nontronite clays, Saponite clays, Sauconite clays and mixtures thereof. Montmorillonite clays are the preferred softening clays. Bentonites comprise mainly montmorillonites and can serve as the preferred source for the fabric-softening clay. The Bentonites can be employed as powder or crystals.

Suitable Bentonites are for example marketed under the trade names Laundrosil® from Süd-Chemie or under the trade name Detercal® from Laviosa.

Suitable cationic polymers particularly include those described in “CTFA International Cosmetic Ingredient Dictionary”, Fourth Edition, J. M. Nikitakis, et al, Editors, published by the Cosmetic, Toiletry, and Fragrance Association, 1991 and grouped under the general term “polyquatemium”. Some suitable polyquatemium compounds are listed below in more detail.

POLYQUATERNIUM-1 (CAS-Number: 68518-54-7)

Definition: {(HOCH₂CH₂)₃N⁺—CH₂CH═CHCH₂—[N⁺(CH₃)₂—CH₂CH═CHCH₂]ₓ—N⁺(CH₂CH₂OH)₃}[Cl⁻]ₓ₊₂

POLYQUATERNIUM-2 (CAS-Number: 63451-27-4)

Definition: [—N(CH₃)₂—CH₂CH₂CH₂—NH—C(O)—NH—CH₂CH₂CH₂—N(CH₃)₂—CH₂CH₂OCH₂CH₂—]²⁺(Cl⁻)₂

Available for example as Mirapol® A-15 (ex Rhodia)

(POLYQUATERNIUM-3)

Definition: copolymer of acrylamide and trimethylammoniumethyl methacrylate methosulfate

POLYQUATERNIUM-4 (CAS-Number: 92183-41-0)

Definition: copolymer of hydroxyethylcellulose and diallyldimethylammonium chloride

Available, for example, as Celquat® H 100 or Celquat® L200 (ex National Starch)

POLYQUATERNIUM-5 (CAS-Number: 26006-22-4)

Definition: copolymer of acrylamide and β-methacrylyloxyethyltrimethylammonium methosulfate. Available for example as Nalco 7113 (ex Nalco) or Reten® 210, Reten® 220, Reten® 230, Reten® 240, Reten® 1104, Reten® 1105 or Reten® 1106 (all ex Hercules)

POLYQUATERNIUM-6 (CAS-Number: 26062-79-3)

Definition: polymer of dimethyldiallylammonium chloride.

Available for example as Merquat® 100 (ex Ondeo-Nalco)

POLYQUATERNIUM-7 (CAS-Number: 26590-05-6)

Definition: polymeric quaternary ammonium salt consisting of acrylamide and dimethyldiallylammonium chloride monomers.

Available for example as Merquat® 550 or Merquat® S (ex Ondeo-Nalco) (POLYQUATERNIUM-8)

Definition: polymeric quaternary ammonium salt of methyl and stearyl dimethylaminoethyl methacrylate, quaternized with dimethyl sulfate.

(POLYQUATERNIUM-9)

Definition: polymeric quaternary ammonium salt of polydimethylaminoethyl methacrylate, quaternized with methyl bromide.

POLYQUATERNIUM-10 (CAS-Numbers: 53568-66-4; 55353-19-0; 54351-50-7; 81859-24-7; 68610-92-4; 81859-24-7)

Definition: polymeric quaternary ammonium salt of hydroxyethyl cellulose, reacted with a trimethylammonium substituted epoxide.

Available for example as Celquat® SC-240 (ex National Starch), UCARE® Polymer JR-125, UCARE® Polymer JR-400, UCARE® Polymer JR-30M, UCARE® Polymer LR 400, UCARE® Polymer LR 30M, Ucare® Polymer SR-10 (all ex Amerchol)

POLYQUATERNIUM-11 (CAS-Number: 53633-54-8)

Definition: quaternary ammonium polymer formed by reacting the copolymer of vinyl pyrrolidone and dimethylaminoethyl methacrylate with diethyl sulfate. Available for example as Luviquat®PQ 11 PN (ex BASF), Gafquat® 734, Gafquat® 755 or Gafquat® 755N (ex GAF)

POLYQUATERNIUM-12 (CAS-Number: 68877-50-9)

Definition: quaternary ammonium polymer salt, obtained by reacting ethyl methacrylate/abietyl methacrylate/diethylaminoethyl methacrylate copolymer with dimethyl sulfate

POLYQUATERNIUM-13 (CAS Number: 68877-47-4)

Definition: polymeric quaternary ammonium salt, obtained by reacting ethyl methacrylate/oleyl methacrylate/diethylaminoethyl methacrylate copolymer with dimethyl sulfate

POLYQUATERNIUM-14 (CAS-Number: 27103-90-8)

Definition: polymeric quaternary ammonium salt of the Formula -{—CH₂—C—(CH₃)—[C(O)O—CH₂CH₂—N(CH₃)₃]}ₓ⁺[CH₃SO₄]⁻ₓ

POLYQUATERNIUM-15 (CAS-Number: 35429-19-7)

Definition: copolymer of acrylamide and

β-methacrylyloxyethyltrimethylammonium chloride

POLYQUATERNIUM-16 (CAS-Number: 95144-24-4)

Definition: polymeric quaternary ammonium salt, formed from methylvinylimidazolium chloride and vinyl pyrrolidone

Available for example as Luviquat® FC 370, Luviquat® Style, Luviquat® FC 550 or Luviquat® Excellence (all ex BASF)

POLYQUATERNIUM-17 (CAS-Number: 90624-75-2)

Definition: polymeric quaternary ammonium salt, obtained by reacting adipic acid and dimethylaminopropylamine with dichloroethyl ether.

Available for example as Mirapol® AD-1 (ex Rhodia)

POLYQUATERNIUM-18

Definition: polymeric quaternary ammonium salt, obtained by reacting azelaic acid and dimethylaminopropylamine with dichloroethyl ether.

Available for example as Mirapol® AZ-1 (ex Rhodia)

POLYQUATERNIUM-19

Definition: polymeric quaternary ammonium salt, obtained by reacting polyvinyl alcohol with 2,3-epoxypropylamine

POLYQUATERNIUM-20

Definition: polymeric quaternary ammonium salt, obtained by reacting polyvinyloctadecyl ether with 2,3-epoxypropylamine.

POLYQUATERNIUM-21 (CAS-Number: 102523-94-4)

Definition: polysiloxane/polydimethyldialkylammonium acetate copolymer

Available for example as Abil® B 9905 (ex Goldschmidt-Degussa)

POLYQUATERNIUM-22 (CAS-Number: 53694-17-0)

Definition: dimethyldiallylammonium chloride/acrylic acid copolymer

Available for example as Merquat® 280 (ex Ondeo-Nalco)

POLYQUATERNIUM-24 (CAS-Number: 107987-23-5)

Definition: polymeric quaternary ammonium salt, obtained by reacting hydroxyethyl cellulose with a lauryldimethylammonium-substituted epoxide

Available for example as Quatrisoft® Polymer LM 200 (ex Amerchol)

POLYQUATERNIUM-27

Definition: block copolymer from the reaction of polyquaternium-2 with polyquaternium-17.

POLYQUATERNIUM-28 (CAS-Number: 131954-48-8)

Definition: vinyl pyrrolidone/methacrylamidopropyltrimethylammonium chloride copolymer

Available for example as Gafquat® HS-100 (ex GAF)

POLYQUATERNIUM-29

Definition: chitosan reacted with propylene oxide and quaterized with epichlorohydrin

POLYQUATERNIUM-30

Definition: polymeric quaternary ammonium salt of the Formula —[CH₂C(CH₃)(C(O)OCH₃)]ₓ—[CH₂C(CH₃)(C(O)OCH₂CH₂N⁺(CH₃)₂CH₂COO⁻)]_{y}—

POLYQUATERNIUM-31 (CAS-Number. 136505-02-7)

POLYQUATERNIUM-32 (CAS-Number: 35429-19-7)

Definition: polymer of N,N,N-trimethyl-2-[(2-methyl-1-oxo-2-propenyl)oxy]-ethanaminium chloride with 2-propenamide

POLYQUATERNIUM-37 (CAS-Number: 26161-33-1)

Definition: homopolymer of methacryloyltrimethylchloride

Available for example as Synthalen® CR (ex 3V Sigma)

POLYQUATERNIUM-44 (CAS-Number: 150595-70-5)

Definition: quaternary ammonium salt of the copolymer of vinyl pyrrolidone and quaternized imidazoline

Available for example as Luviquat® Ultracare (ex BASF)

POLYQUATERNIUM-68 (CAS-Number: 827346-45-2)

Definition: quaternized copolymer of vinyl pyrrolidone, methacrylamide, vinyl imidazole and quaternized vinyl imidazole

Available for example as Luviquat® Supreme (ex BASF)

Besides the already mentioned Polyquatemium-10, other quaternized celluloses, quaternized cellulose derivatives, amine-modified celluloses or amine-modified cellulose derivatives also represent suitable cationic polymers.

It can be preferred that the fragrance-transmitting composition comprises a fabric-softening compound and one or more additional fabric-care compound(s).

It can also be preferred that the fragrance-transmitting composition is free of cationic fabric-care compounds. In particular, it can be preferred that the fragrance-transmitting composition is free of quaternary ammonium compounds.

The amount of fabric-care compound in the fragrance-transmitting composition, when present, is 0.1 to 15 wt. % and preferably between 2 and 12 wt. %.

It is preferred that the fragrance-transmitting composition is in powder form and is present at least partially in and/or at least partially on the coating.

In order to improve the esthetic impression of the fragrance-transmitters, they may be colored with appropriate colorants. Preferred colorants, which are not difficult for the expert to choose, have high storage stability, are not affected by the other ingredients of the washing or cleaning agent and by light and do not have any pronounced substantivity for textile fibers, so as not to color them.

Moreover, the fragrance-transmitting composition can comprise a filler, such as silica. The amount of filler can range between 0.1 and 10 wt % and preferably ranges from 1 to 5 wt %.

In order to increase the gloss, the fragrance-transmitting composition can also comprise a pearlizer. Exemplary suitable pearlizers are ethylene glycol mono and distearate (for example Cutina® AGS from Cognis) as well as PEG-3 -distearate.

In addition, the fragrance-transmitting composition can contain a skin-care compound.

A skin-care compound is understood to mean a compound or a mixture of compounds that on contact of a fabric with the washing agent is absorbed onto the fabric, and on contact of the fabric with skin lends the skin an advantage compared with a fabric that was not treated with the washing and cleaning agent according to the invention. This advantage can include for example the transfer of the skin-care compound from the fabric onto the skin, a lower water transfer from the skin to the fabric or a lower friction between the surface of the skin and the fabric.

The skin-care compound is preferably hydrophobic, can be liquid or solid and must be compatible with the other ingredients of the solid fragrance-transmitting composition. The skin-care compound can contain for example
a) waxes such as carnuba, spermaceti, beeswax, lanolin, derivatives thereof as well as their mixtures;
b) plant extracts, for example vegetal oils such as avocado oil, olive oil, palm oil, palm nut oil, rape seed oil, linseed oil, soya oil, peanut oil, coriander oil, castor oil, poppy-seed oil, cocoa oil, coconut oil, pumpkin seed oil, wheat germ oil, sesame oil, sunflower oil, almond oil, macadamia nut oil, apricot nut oil, hazel nut oil, jojoba oil or canola oil, camomile, aloe vera as well as their mixtures;
c) higher fatty acids such as lauric acid, myristic acid, palmitic acid, stearic acid, behenic acid, oleic acid, linoleic acid, linolenic acid, isostearic acid or polyunsaturated fatty acids;
d) higher fatty alcohols such as lauryl alcohol, cetyl alcohol, stearyl alcohol, oleyl alcohol, behenyl alcohol or 2-hexadecanol,
e) esters, such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate or alkyl tartrate;
f) hydrocarbons such as paraffins, mineral oils, squalane or squalene;
g) lipids;
h) vitamins such as vitamin A, C or E or vitamin alkyl esters;
i) phospholipids;
j) sun protection agents such as octyl methoxylcinnamate and butyl methoxybenzoylmethane;
k) silicone oils such as linear or cyclic polydimethylsiloxanes, amino-, alkyl-, alkylaryl-or aryl-substituted silicone oils and
l) mixtures thereof

The quantity of skin-care compound is preferably between 0.01 and 10 wt %, preferably between 0.1 and 5 wt % and quite particularly preferably between 0.3 and 3 wt % based on the solid fragrance-transmitting composition. It may be the case that the skin-care compound also additionally possesses a fabric-care effect.

For manufacturing a solid fragrance-transmitting composition, the water-soluble carrier is firstly loaded with the perfume and additional ingredients, when present. The saccharide is melted and added to the particulate carrier in such a way that the latter is at least partially coated. The solid fragrance-transmitting composition is preferably completely coated with the saccharide.

In order to vary the degree of hardness of the saccharide coating, it can be advantageous to add additional ingredients, such as water and/or binder to the saccharide melt. Anionic surfactants, including soaps, non-ionic surfactants, cationic surfactants, betaines, urea (derivatives), water glass, polyethylene glycols, glycerin, starches as well as mixtures thereof can be the binders. The amount of water and/or binder is preferably 20 wt % at most, based on the total coating material (“saccharide glaze”). Consequently, the saccharide coating can additionally comprise water and/or binder. The addition of binder in the coating leads to a homogenization and stabilization of the saccharide glazes. In the context of this application, the term saccharide glaze is understood to mean the saccharide and optionally water and/or binder coating that has been deposited onto the carrier loaded with perfume. The presence of binder in the saccharide glaze also makes the glaze less sensitive towards mechanical stress and/or temperature influences.

In another preferred embodiment, the solid fragrance-transmitting composition possesses a powdered fabric-care compound. This can be present at least partially in the coating with saccharide. For this the powdered fabric-care compound is at least partially incorporated into the still not completely solidified saccharide coating deposited on the carrier. Alternatively, after the coating has solidified it can be coated with the powdered fabric-care compound.

In a quite particularly preferred embodiment of the invention, a mixture of the powdered fabric-care compound and a polysaccharide is incorporated or the coating is coated with such a mixture.

Solid fragrance-transmitting compositions, in which a powdered fabric-care compound or a mixture of a powdered fabric-care compound and a polysaccharide is present at least partially in and/or at least partially on the coating, show no or only quite slight tendencies to clump together or agglomerate during manufacture and/or storage.

In order to manufacture a solid fragrance-transmitting composition, a carrier is first placed in a pre-heated mixer. In the mixing process the carrier is then loaded with perfume and optionally with additional ingredients. A saccharide-containing melt is then uniformly deposited onto the perfumed carrier and left to solidify. If the solid fragrance-transmitting composition comprises a powdered fabric-care compound or a mixture of a fabric-care compound and a polysaccharide, then the coated, perfumed carrier particles are dusted with it, as long as the saccharide-containing melt is still not completely solidified.

The perfume, the additional ingredients and the saccharide-containing melt are preferably deposited by means of spraying or dusting, such that they are preferably added in liquid, dissolved and/or dispersed form.

The solid fragrance-transmitting composition is particularly suitable for perfuming, or perfuming and conditioning textile fabrics and for this is brought into contact with the textile fabrics in the rinse cycle of a conventional washing and cleaning process.

The fragrance-transmitting composition can be incorporated into a washing or cleaning agent. For this, a solid washing or cleaning agent is mixed with 0,1 to 20 wt %, preferably 1 to 10 wt % of the fragrance-transmitting composition according to the invention.

However, the solid fragrance-transmitting composition is preferably added separately in the rinse cycle.

### EXAMPLES

In Table 1 are shown inventive fragrance-transmitting compositions E1 to E3 (all quantities are given in wt % active substance, based on the composition).

TABLE 1

| | E1 | E2 | E3 |
|---|---|---|---|
| NaCI crystals (1 to 3 mm) | 79 | — | — |
| Saccharose crystals (1-4 mm) | — | 69 | — |
| Citric acid crystals (1-3 mm) | — | | 69 |
| Bentonite (powder) | — | 10 | 10 |
| Perfume | 1 | 1 | 1 |
| Saccharide-containing coating | 20 | 20 | 20 |

In Table 2 is shown the composition of the saccharide-containing coating (all quantities are given in wt % active substance, based on the composition).

TABLE 2

| | |
|---|---|
| Saccharose powder | 84.99 |
| C₁₂-C₁₈ ROH (7 EO) | 5 |
| Colorant | 0.01 |
| Water | 10 |

For the manufacture of the fragrance-transmitting composition E1 the NaCl crystals were placed in a Lödige mixer preheated to 40 to 60° C. A saccharide-containing melt was prepared separately, in that the saccharose was dissolved in hot water and the non-ionic surfactant and the colorant was added. First the perfume and then the saccharide-containing melt were then sprayed on. After cooling the melt, a solid fragrance-transmitting composition with a crystal-like visual appearance was obtained which is dissolved in the rinse cycle of a washing machine without leaving a residue.

For manufacturing the fragrance-transmitting compositions E2 and E3, in addition Bentonite powder was dusted on to the already deposited saccharide-containing melt before the melt had completely solidified. In this case as well, a solid fragrance-transmitting composition with a crystal-like visual appearance was obtained which is dissolved in the rinse cycle of a washing machine without leaving a residue. No deposits/residues at all were observed in the dispensing draw of the washing machine.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A solid fragrance-transmitting composition comprising a particulate water-soluble carrier, and perfume, wherein the composition has at least partial coating of a saccharide, wherein the saccharide is selected from the group consisting of monosaccharides, oligosaccharides and mixtures thereof.

2. The solid fragrance-transmitting composition according to claim 1, wherein the water-soluble carrier is selected from the group consisting of inorganic alkali metal salts, organic alkali metal salts, inorganic alkaline earth metal salts, organic alkaline earth metal salts, organic acids, carbohydrates, silicates, urea and mixtures thereof.

3. The solid fragrance-transmitting composition according to claim 1, wherein the amount of the water-soluble carrier is 50 to 99 wt % based on the total solid fragrance-transmitting composition.

4. The solid fragrance-transmitting composition according to claim 1, wherein the solid fragrance-transmitting composition additionally possesses an ingredient selected from the group of the fabric-care compounds, colorants, fillers, pearlizers, skin-care compounds and mixtures thereof.

5. The solid fragrance-transmitting composition according to claim 4, wherein the fabric-care compound is selected from fabric-softening compounds, bleaching agents, bleach activators, enzymes, silicone oils, anti-redeposition agents, optical brighteners, graying inhibitors, shrink inhibitors, anti-creasing agents, color transfer inhibitors, antimicrobials, germicides, fungicides, antioxidants, antistats, ironing auxiliaries, water proofing and impregnation agents, swelling and anti-slip agents, UV absorbers and mixtures thereof.

6. The solid fragrance-transmitting composition according to claim 5, wherein the fabric-care compound is a fabric-softening compound.

7. The solid fragrance-transmitting composition according to claim 6, wherein the fabric-softening compound is selected from quaternary ammonium compounds, polysiloxanes, fabric-softening clays, cationic polymers and mixtures thereof.

8. The solid fragrance-transmitting composition according to claim 4, wherein the fabric-care compound is in powder form and is present at least partially in and/or at least partially on the coating.

9. The solid fragrance-transmitting composition according to claim 8, wherein, in addition to the fabric-care compound, a polysaccharide is present at least partially in and/or at least partially on the coating.

10. The solid fragrance-transmitting composition according to claim 1, wherein the saccharide is saccharose.

11. The solid fragrance-transmitting composition according to claim 1, wherein the amount of perfume is 0.1 to 20 wt %.

12. The solid fragrance-transmitting composition according to claim 1, wherein the particle size of the water-soluble carrier is in the range 0.6 to 30 mm.

13. The method of perfuming and/or conditioning textile fabrics wherein the solid composition according to claim 1 is brought into contact with the textile fabrics in a rinse cycle of a conventional washing process.

14. A process for manufacturing a solid fragrance-transmitting composition comprising a particulate water-soluble carrier, and perfume, wherein the composition has at least partial coating of a saccharide, wherein the saccharide is selected from the group of monosaccharides, oligosaccharides and mixtures thereof, in which the water-soluble carrier is treated with the perfume, the saccharide is melted and is deposited in such a way onto the particulate carrier such that said carrier is at least partially coated.
